(19) United States
(12) Patent Application Publication
(10) Pub. No.: US 2019/0282476 A1
(43) Pub. Date: Sep. 19, 2019

(54) Cleansing Composition

(71) Applicant: Colgate-Palmolive Company, New York, NY (US)

(72) Inventors: Huiyan YIN, Guangzhou (CN); Xiang Dong HE, Guangzhou (CN)

(73) Assignee: Colgate-Palmolive Company, New York, NY (US)

(21) Appl. No.: 16/065,957

(22) PCT Filed: Dec. 29, 2015
(86) PCT No.: PCT/CN2015/099481

§ 371 Date: Jun. 25, 2018

(51) Int. Cl. A61K 8/368; A61K 8/46; A61K 8/44; A61K 8/73; A61K 8/49; A61K 8/34; A61K 8/362; A61Q 19/10

(52) U.S. Cl. CPC A61K 8/368; A61K 8/463; A61K 8/442; A61K 8/73; A61Q 19/10; A61K 8/4946; A61K 8/4986; A61K 8/347; A61K 8/362; A61K 8/731

(57) ABSTRACT

Described herein, are cleansing compositions comprising a surfactant, phenoxyethanol, sodium salicylate, a polydentate chelating agent, and a structuring agent selected from: a gum and a polyacrylate.

### BACKGROUND

When cleansing compositions are prepared, they need to be preserved to prevent bacterial growth in the composition. Preservatives have been used in cleansing compositions in the past. Examples of preservatives include parabens, DMDM hydantoin, halogenated diphenyl ethers, isothiazolinones, benzalkonium chloride, and benzethonium chloride. However, there is also a desire to formulate products with other preservative agents, which may impact efficacy, stability or consumer appeal (e.g., texture, color, or smell). As such, there remains a need for products having alternative preservative agents, which provide the desired efficacy, stability and/or consumer appeal. Some embodiments of the present invention are designed to meet these needs.

### BRIEF SUMMARY

Provided is a preserved cleansing composition comprising surfactant, phenoxyethanol, sodium salicylate, and a polydentate chelating agent (e.g., a salt of ethylenediaminetetraacetic acid), and wherein (a) the composition further comprises a structuring agent chosen from gum and polyacrylate, and/or (b) the composition comprises a weight ratio of phenoxyethanol to sodium salicylate of 3:1 to 3.7:1. The phenoxyethanol, sodium salicylate, and the salt of ethylenediaminetetraacetic acid are able to preserve the surfactant cleansing composition.

Further areas of applicability of the present invention will become apparent from the detailed description provided hereinafter. It should be understood that the detailed description and specific examples, while indicating the preferred embodiment of the invention, are intended for purposes of illustration only and are not intended to limit the scope of the invention.

### DETAILED DESCRIPTION

As used throughout, ranges are used as shorthand for describing each and every value that is within the range. Any value within the range can be selected as the terminus of the range. In addition, all references cited herein are hereby incorporated by referenced in their entireties. In the event of a conflict in a definition in the present disclosure and that of a cited reference, the present disclosure controls.

Unless otherwise specified, all percentages and amounts expressed herein and elsewhere in the specification should be understood to refer to percentages by weight. The amounts given are based on the active weight of the material.

In one embodiment, provided is a Cleansing Composition 1 comprising surfactant, phenoxyethanol, sodium salicylate, and polydentate chelating agent (e.g., a salt of ethylenediaminetetraacetic acid), and wherein (a) the composition further comprises a structuring agent chosen from gum and polyacrylate, and/or (b) the composition comprises a weight ratio of phenoxyethanol to sodium salicylate of 3:1 to 3.7:1.
1.1 Cleansing Composition 1, wherein the composition further comprises a structuring agent chosen from gum and polyacrylate.
1.2 Cleansing Composition 1 or 1.1, wherein the composition comprises a weight ratio of phenoxyethanol to sodium salicylate of 3:1 to 3.7:1.
1.3 Cleansing Composition 1 or 1.1 et seq., wherein phenoxyethanol is present in an amount of 0.5 to 3% by weight of the composition.
1.4 Cleansing Composition 1 or 1.1 et seq, wherein the phenoxyethanol is present at 0.8 to 3% by weight of the composition, optionally 0.8 to 1.2% by weight of the composition or about 1% by weight of the composition.
1.5 Cleansing Composition 1 or 1.1 et seq, wherein the sodium salicylate is present in an amount of 0.1 to 1% by weight of the composition.
1.6 Cleansing Composition 1 or 1.1 et seq, wherein the sodium salicylate is present in an amount of 0.2 to 1% by weight of the composition, optionally 0.2 to 0.4% by weight of the composition or about 0.3% by weight of the composition.
1.7 Cleansing Composition 1 or 1.1 et seq, wherein the weight ratio of phenoxyethanol to sodium salicylate is from 3.0:1 to 3.6:1, or from 3.1:1 to 3.5:1, or 3.2:1 to 3.4:1, or about 3.3:1.
1.8 Cleansing Composition 1 or 1.1 et seq, wherein the polydentate chelating agent is selected from a bidentate, tridentate, tetradentate, pentadentate or hexadentate chelating agent.
1.9 Cleansing Composition 1 or 1.1 et seq, wherein the polydentate chelating agent is a hexadentate chelating agent.
1.10 Cleansing Composition 1 or 1.1 et seq, wherein the chelating agent is an aminopolycarboxylic acid, or salt thereof
1.11 Cleansing Composition 1 or 1.1 et seq, wherein the chelating agent is selected from the group consisting of ethylenediaminetetracetic acid, iminodisuccinic acid, polyaspartic acid, polyglutamic acid, ethylenediamine-N,N′-disuccinic acid, methylglycineacetic acid, and glutamic acid N,N′-diacetic acid, any of which can be in free or salt form, including mixed acid/salt form and mixed salt form.
1.12 Cleansing Composition 1 or 1.1 et seq, wherein the chelating agent is a salt of ethylenediaminetetraacetic acid.
1.13 Cleansing Composition 1 or 1.1 et seq, wherein the chelating agent (e.g., the salt of ethylenediaminetetraacetic acid) is present in an amount of 0.05 to 0.5% by weight of the composition.
1.14 Cleansing Composition 1 or 1.1 et seq, wherein the chelating agent (e.g., the salt of ethylenediaminetetraacetic acid) is present in an amount of 0.1 to 0.5% by weight of the composition, optionally 0.1 to 0.2% by weight of the composition or about 0.13% by weight of the composition.
1.15 Cleansing Composition 1 or 1.1 et seq, wherein chelating agent is tetrasodium ethylenediaminetetraacetate (tetrasodium EDTA).
1.16 Cleansing Composition 1.15, wherein tetrasodium ethylenediaminetetraacetate is the only salt of ethylenediaminetetraacetic acid present in the composition.
1.17 Cleansing Composition 1 or 1.1 et seq, wherein the structuring agent is present in an amount of 0.5 to 5% by weight of the composition.
1.18 Cleansing Composition 1 or 1.1 et seq, wherein the structuring agent is present in an amount of 0.5 to 1.5% by weight of the composition, optionally about 0.9% by weight of the composition.
1.19 Cleansing Composition 1 or 1.1 et seq, wherein the gum is selected from the group consisting of xanthan gum, gellan gum, guar gum, rhamsan gum, furcellaran gum, carrageenan, arabinogalactan, alginate, and pectin.
1.20 Cleansing Composition 1 or 1.1 et seq, wherein the gum is xanthan gum.
1.21 Cleansing Composition 1 or 1.1 et seq, wherein the surfactant comprises sodium laureth sulfate and cocoamidopropyl betaine.
1.22 Cleansing Composition 1 or 1.1 et seq, wherein the surfactant is present in an amount of 1 to 40% by weight of the composition, optionally, 5 to 15% by weight of the composition, e.g., 9 to 12% by weight of the composition, or 10 to 11% by weight of the composition, or about 10% by weight of the composition.
1.23 Cleansing Composition 1 or 1.1 et seq further comprising a hydroxyalkyl cellulose polymer, e.g., hydroxypropyl cellulose, hydroxybutyl cellulose, or hydroxyethyl cellulose, optionally in an amount of 0.1 to 1% by weight of the composition, e.g., 0.1 to 0.5%, or 0.2 to 0.3%, or about 0.24% by weight of the composition.
1.24 Cleansing Composition 1 or 1.1 et seq comprising:
   a) sodium laureth sulfate,
   b) cocoamidopropyl betaine,
   c) 0.5 to 1.5% by weight of the composition of xanthan gum,
   d) 0.8 to 1.2% by weight of the composition of phenoxyethanol,
   e) 0.2 to 0.4% by weight of the composition of sodium salicylate, and
   f) 0.1 to 0.2% by weight of the composition of tetrasodium ethylenediaminetetraacetate.
1.25 Cleansing Composition 1 or 1.1 et seq, wherein the composition does not contain one or more of parabens, DMDM hydantoin, halogenated diphenyl ethers, isothiazolinones, benzalkonium chloride, and benzethonium chloride, for example, wherein the composition does not contain any of parabens, DMDM hydantoin, halogenated diphenyl ethers, isothiazolinones, benzalkonium chloride, and benzethonium chloride.
1.26 Cleansing Composition 1 or 1.1 et seq, wherein phenoxyethanol, sodium salicylate, and a salt of ethylenediaminetetraacetic acid (e.g., tetrasodium EDTA) are the only preservative agents in the composition.
1.27 Cleansing Composition 1 or any of 1.1 to 1.26, wherein water is present in the composition in an amount of 50 to 90% by weight of the composition, optionally 80 to 90% by weight of the composition.

The cleansing compositions, e.g. any of Composition 1 or 1.1 to 1.27, include as preservatives, phenoxyethanol, sodium salicylate and a chelating agent (e.g., a salt of ethylenediaminetetraacetic acid). It has been found that in some commercially available personal care compositions comprising sodium salicylate, the measurable amount of sodium salicylate is very low (less than 0.01% by weight). It is well known that preservative agents have established minimum inhibitory concentrations, and below such concentrations preservative agents are not effective in preventing microbial growth. It has been found that for typical microorganisms encountered in personal care compositions, the MIC for sodium salicylate is about 0.019% by weight.

It has been found that the use of a polydentate chelating agent, such as a salt of ethylenediaminetetraacetic acid, enhances the effectiveness of the preservative combination of phenoxyethanol and sodium salicylate. Without being bound by theory, it is believed that the presence of a chelating agent may prevent adverse reaction between trace metal ions in the composition and salicylate ion. For example, ions such as iron (Fe(III)), calcium, magnesium, and manganese(II) may interact with salicylate to cause the formation of insoluble complexes which may precipitate out of the composition. This can result in a loss of preservative efficacy. Chelating agents may prevent this occurrence by chelating the trace metal ions that are present in the composition. In addition, such chelating agents may also complex with ions that are important to microbial growth and metabolism, thus directly exerting some preservative effect as well. For example, chelating agents may complex with metal ions that are important for bacterial or fungal metalloproteinase enzyme function.

The chelating agent used in the compositions of the present disclosure can be any suitable chelating agents which are generally recognized as safe for personal care compositions. In some embodiments, the chelating agents are polydentate chelating agents, for example, selected from bidentate, tridentate, tetradentate, pentadentate or hexadentate chelating agents. In some embodiments, the chelating agent is an aminopolycarboxylic acid, for example, a hexadentate aminopolycarboxylic acid. Suitable chelating agents include, but are not limited to ethylenediaminetetracetic acid, iminodisuccinic acid, polyaspartic acid, polyglutamic acid, ethylenediamine-N,N′-disuccinic acid, methylglycineacetic acid, and glutamic acid N,N′-diacetic acid, any of which can be in free or salt form, including mixed acid/salt form and mixed salt form. Any suitable salts can be used, including sodium, potassium and calcium salts, or mixed salts, or mixtures thereof.

In some embodiments, the chelating agent is a salt of ethylenediaminetetraacetic acid. The salt can be any suitable salt, for example, the tetrasodium salt, the disodium salt, the tetrapotassium salt, the dipotassium salt, the dicalcium salt or mixed salts (e.g., the calcium disodium salt).

The cleansing compositions, e.g. any of Composition 1 or 1.1, include one or more anionic surfactants, amphoteric surfactants, nonionic surfactants, cationic surfactants, and combinations thereof. Surfactants can be included in any desired amount. In some embodiments, surfactants are present in the composition in an amount of 1 to 40% by weight, e.g., in an amount of 5 to 40% by weight, or in an amount of 5 to 15% by weight, or in an amount of 9 to 12% by weight, or 10 to 11% by weight, or about 10% by weight.

A variety of anionic surfactants can be utilized including, for example, long chain alkyl (C₆-C₂₂) materials such as long chain alkyl sulfates, long chain alkyl sulfonates, long chain alkyl phosphates, long chain alkyl ether sulfates, long chain alkyl alpha olefin sulfonates, long chain alkyl taurates, long chain alkyl isethionates (SCI), long chain alkyl glyceryl ether sulfonates (AGES), sulfosuccinates and the like. These anionic surfactants can be alkoxylated, for example, ethoxylated, although alkoxylation is not required. These surfactants are typically highly water soluble as their sodium, potassium, alkyl and ammonium or alkanol ammonium containing salt form and can provide high foaming cleansing power. Other equivalent anionic surfactants may be used. In one embodiment, the anionic surfactant comprises sodium laureth sulfate, sodium pareth sulfate, and combinations thereof. Anionic surfactants can be included in any desired amount. In one embodiment, anionic surfactants are present in the composition in an amount of 0 to 30% by weight.

Amphoteric surfactants may also be included in the composition. These surfactants are typically characterized by a combination of high surfactant activity, lather forming and mildness. Amphoteric surfactants include, but are not limited to, derivatives of aliphatic secondary and tertiary amines in which the aliphatic radical can be straight chain or branched and wherein one of the aliphatic substituents contains 8 to 18 carbon atoms and one contains an anionic water solubilizing group, e.g., carboxy, sulfonate, sulfate, phosphate, or phosphonate. Examples of such compounds include sodium 3-dodecyaminopropionate, sodium 3-dodecylaminopropane sulfonate, N-alkyl taurines and N-higher alkyl aspartic acids. Other equivalent amphoteric surfactants may be used. Examples of amphoteric surfactants include, but are not limited to, a range of betaines including, for example, high alkyl betaines, such as coco dimethyl carboxymethyl betaine, lauryl dimethyl carboxy-methyl betaine, lauryl dimethyl alpha-carboxyethyl betaine, cetyl dimethyl carboxymethyl betaine, lauryl bis-(2-hydroxyethyl)carboxy methyl betaine, stearyl bis-(2-hydroxypropyl)carboxymethyl betaine, oleyl dimethyl gamma-carboxypropyl betaine, and lauryl bis-(2-hydroxypropyl)alpha-carboxyethyl betaine, sulfobetaines such as coco dimethyl sulfopropyl betaine, stearyl dimethyl sulfopropyl betaine, amido betaines, amidosulfobetaines and the like. Betaines having a long chain alkyl group, particularly coco, may be particularly useful as are those that include an amido groups such as the cocamidopropyl and cocoamidoethyl betaines. Amphoteric surfactants can be included in any desired amount. In one embodiment, amphoteric surfactants are present in the composition in an amount of 0 to 25% by weight.

Examples of nonionic surfactants include, but are not limited to, polysorbate 20, long chain alkyl glucosides having C₈-C₂₂ alkyl groups; coconut fatty acid monoethanolamides such as cocamide MEA; coconut fatty acid diethanolamides, fatty alcohol ethoxylates (alkylpolyethylene glycols); alkylphenol polyethylene glycols; alkyl mercaptan polyethylene glycols; fatty amine ethoxylates (alkylaminopolyethylene glycols); fatty acid ethoxylates (acylpolyethylene glycols); polypropylene glycol ethoxylates (for example the PLURONIC™ block copolymers commercially available from BASF); fatty acid alkylolamides, (fatty acid amide polyethylene glycols); N-alkyl-, N-alkoxypolyhydroxy fatty acid amides; sucrose esters; sorbitol esters; polyglycol ethers; and combinations thereof. Nonionic surfactants can be included in any desired amount. In one embodiment, nonionic surfactants are present in the composition in an amount of 0 to 15% by weight.

Cationic surfactants can also be included in the composition. Examples of cationic surfactants include, but are not limited to quaternium and polyquaternium compounds. Cationic surfactants can be included at any desired level. In one embodiment, cationic surfactants are present in the composition in an amount of 0 to 10% by weight.

In one embodiment, the surfactants comprise sodium laureth sulfate and cocoamidopropyl betaine. In one embodiment, the sodium laureth sulfate is present in an amount of 5 to 12%, e.g. 8 to 9% by weight of the composition, and the cocoamidopropyl betaine is present in an amount of 1 to 2.5%, e.g. 1.5 to 2.0%, by weight of the composition.

The composition includes a structuring agent chosen from gum and polyacrylate. The gum can be chosen from xanthan gum, gellan gum, guar gum, rhamsan gum, furcellaran gum, carrageenan, arabinogalactan, alginate, and pectin. Examples of the polyacrylate include, but are not limited to, polyacrylate aqueous solutions manufactured by Lubrizol as CARBOPOL™ resins, also known as CARBOMER™, which are hydrophilic high molecular weight, crosslinked acrylic acid polymers. In one embodiment, the polymer is CARBOPOL™ Aqua SF-1 or Aqua SF-2. Other polymers that can be used include, but are not limited to, CARBOPOL™ Aqua 30, CARBOPOL™ 940 with a molecular weight of approximately 4,000,000, and CARBOPOL™ 934 with a molecular weight of approximately 3,000,000. In one embodiment, the structuring agent is xanthan gum. In one embodiment, the structuring agent is present in an amount of 0.5 to 5% by weight of the composition. In other embodiments, the structuring agent is present in an amount of 0.5 to 1.5% by weight of the composition, optionally about 0.9% by weight of the composition.

In some embodiments, the cleansing composition can further include a hydroxyalkyl cellulose polymer, e.g., hydroxypropyl cellulose, hydroxybutyl cellulose, or hydroxyethyl cellulose. In certain embodiments, the hydroxyalkyl cellulose, e.g. hydroxyethyl cellulose, is present in an amount of 0.1 to 1% by weight of the composition e.g., 0.1 to 0.5%, or 0.2 to 0.3%, or about 0.24%.

Water may be included in the composition. Water can be included in an amount of 30 to 95% by weight of the composition, for example, 50% to 90% by weight of the composition, optionally 80 to 90% by weight of the composition.

In other embodiments, the composition may include any of the following materials in any desired amount to achieve a desired effect in the composition (amounts that can be used in some embodiments are provided): one or more salts, for example, sodium chloride, sodium sulfate, sodium carbonate, sodium bicarbonate and/or their equivalents (0 to 5% by weight); foaming agents, for example decyl glucoside, and/or their equivalents (0 to 3% by weight); glyceryl esters and derivatives, for example glycol distearate, and/or their equivalents(0 to 3%; by weight); organic acids, for example, lactic acid, citric acid and/or formic acid and/or their equivalents (0 to 2% by weight); fragrances and/or perfumes (0 to 5% by weight); pearlizing agents, for example, glycol distearic esters, such as ethylene glycol distearate, but also fatty acid monoglycol esters (0 to 3% by weight); stabilizers, for example, metal salts of fatty acids, such as e.g., magnesium stearate, aluminum stearate and/or zinc stearate (0 to 2% by weight); and dyes and pigments that are approved and suitable for cosmetic purposes. By “their equivalents” is meant compounds with similar structure and properties (e.g., the same anion with a different cation, or the same ester with an alkyl chain of alternative length, or the same acid alternatively in salt form).

In some embodiments, the cleansing composition does not contain one or more of: parabens, DMDM hydantoin, halogenated diphenyl ethers, isothiazolinones, benzalkonium chloride, benzethonium chloride. In some embodiments, the cleansing composition does not contain any of the aforementioned agents. In some embodiments, phenoxyethanol, sodium salicylate and a salt of ethylenediaminetetraacetic acid (e.g., tetrasodium EDTA) are the only preservative agents in the composition.

In some embodiments, the cleansing composition can be used to clean skin or hair.

Other embodiments provide compositions which can be used to clean, inter alia, hard surfaces.

### EXAMPLES

### Example 1

The body wash formulas in Table 1 below are prepared. A comparative formula is preserved with DMDM hydantoin and Kathon™ CG isothiazolinones, and an exemplary composition of the present invention (“Ex. 1”) is preserved with a combination of phenoxyethanol, sodium salicylate, and tetrasodium EDTA.

TABLE 1

| Material | Comparative | Ex. 1 |
|---|---|---|
| Water and minors (color, fragrance, pH, etc.) | Q.S. | Q.S. |
| Sodium laureth sulfate | 8.75 | 8.75 |
| Cocoamidopropyl betaine | 1.7 | 1.7 |
| Xanthan Gum | 0.9 | 0.9 |
| Hydroxyethyl cellulose | 0.24 | 0.24 |
| DMDM Hydantoin | 0.45 | — |
| Isothiazolinone preservative | 0.07 | — |
| Phenoxyethanol | — | 1 |
| Sodium salicylate | — | 0.3 |
| Tetrasodium EDTA | 0.13 | 0.13 |
| MRI | 0.99 | 1.2 |

The Micro Robustness Index (MRI) of the comparative formula and Ex. 1 is evaluated. The Micro Robustness Index is used as a quantitative measure of a composition's ability to withstand microbial challenge. The MRI is the result from a challenge test assessing the antimicrobial efficacy of a compound/composition against a pool of microorganisms including Burkholderia cepacia, Enterobacter cloacae, Escherichia coli, Klebsiella oxytoca, Klebsiella pneumoniae, Serratia marcescens, Providencia rettgeri, Pseudomonas aeruginosa, Pseudomonas putida, Staphylococcus aureus, and Staphylococcus saprophyticus. Samples are challenged 3 times at 30 minute intervals with an inoculum of 10⁷ bacteria from the above listed pool. After 4, 6 and 24 hours, aliquots are tested to measure the log reduction of bacteria. Using this data, the area under the curve (AUC) is calculated and then converted into the MRI score; the higher the MRI, the greater the micro robustness of the tested composition. It has been found that an MRI of at least 0.75 is required in order to show that a composition has an acceptable level of robustness against microbial attack. MRI lower than 0.75 may not adequately reduce the pool of microorganisms and may result in greater risk of microbial attack, for example, spoilage, of the composition.

As shown in Table 1 above, Example 1 is able to effectively preserve the composition, and demonstrated superior micro-robustness to the comparative formula.

What is claimed is:

1. A cleansing composition comprising a surfactant, phenoxyethanol, sodium salicylate, a polydentate chelating agent, and a structuring agent selected from: a gum and a polyacrylate.

2. The cleansing composition of claim 1, wherein the weight ratio of phenoxyethanol to sodium salicylate is from about 3:1 to about 3.7:1.

3. The cleansing composition of claim 1, wherein phenoxyethanol is present in an amount of 0.5 to 3% by weight of the composition.

4. The cleansing composition of claim 1, wherein the phenoxyethanol is present at 0.8 to 3% by weight of the composition, optionally 0.8 to 1.2% by weight of the composition or about 1% by weight of the composition.

5. The cleansing composition of claim 1, wherein the sodium salicylate is present in an amount of 0.1 to 1% by weight of the composition.

6. The cleansing composition of any preceding claim 1, wherein the sodium salicylate is present in an amount of 0.2 to 1% by weight of the composition, optionally 0.2 to 0.4% by weight of the composition or about 0.3% by weight of the composition.

7. The cleansing composition of claim 1, wherein the weight ratio of phenoxyethanol to sodium salicylate is from 3.1:1 to 3.5:1, optionally from 3.2:1 to 3.4:1, or optionally about 3.3:1.

8. The cleansing composition of claim 1, wherein the polydentate chelating agent is a hexadentate chelating agent, optionally, wherein the chelating agent is a salt of ethylenediaminetetraacetic acid.

9. The cleansing composition of claim 1, wherein the chelating agent is present in an amount of 0.05 to 0.5% by weight of the composition.

10. The cleansing composition of claim 1, wherein the chelating agent is present in an amount of 0.1 to 0.5% by weight of the composition, optionally 0.1 to 0.2% by weight of the composition or about 0.13% by weight of the composition.

11. The cleansing composition of claim 1, wherein the chelating agent is tetrasodium ethylenediaminetetraacetate.

12. The cleansing composition of claim 1, wherein tetrasodium ethylenediaminetetraacetate is the only salt of ethylenediaminetetraacetic acid present in the composition.

13. The cleansing composition of claim 1, wherein the structuring agent is present in an amount of 0.5 to 5% by weight of the composition.

14. The cleansing composition of any preceding claim 1, wherein the structuring agent is present in an amount of 0.5 to 1.5% by weight of the composition, optionally about 0.9% by weight of the composition.

15. The cleansing composition of claim 1, wherein the gum is selected from the group consisting of xanthan gum, gellan gum, guar gum, rhamsan gum, furcellaran gum, carrageenan, arabinogalactan, alginate, and pectin.

16. (canceled)

17. The cleansing composition of claim 1, wherein the surfactant comprises sodium laureth sulfate and cocoamidopropyl betaine.

18. The cleansing composition of claim 1, wherein the surfactant is present in an amount of 1 to 40% by weight of the composition, optionally, 5 to 15% by weight of the composition.

19. The cleansing composition of claim 1 further comprising a hydroxyalkyl cellulose, optionally hydroxyethyl cellulose, further optionally in an amount of 0.1 to 1% by weight of the composition.

20. A cleansing composition comprising:
a. sodium laureth sulfate,
b. cocoamidopropyl betaine,
c. 0.5 to 1.5% by weight of the composition of xanthan gum,
d. 0.8 to 1.2% by weight of the composition of phenoxyethanol,
e. 0.2 to 0.4% by weight of the composition of sodium salicylate, and
f. 0.1 to 0.2% by weight of the composition tetrasodium ethylenediaminetetraacetate.

21. The cleansing composition of claim 1, wherein the composition does not contain one or more of parabens, DMDM hydantoin, halogenated diphenyl ethers, isothiazolinones, benzalkonium chloride, and benzethonium chloride.
